(19) United States
(12) Patent Application Publication
(10) Pub. No.: US 2020/0015987 A1
(43) Pub. Date: Jan. 16, 2020

(54) STENT ASSEMBLY AND METHOD OF MANUFACTURING

(71) Applicant: Medibrane Ltd., Rosh Hayin (IL)

(72) Inventors: Elad Einav, Salit (IL); Amir Kraitzer, Herzliya (IL)

(21) Appl. No.: 16/508,990

(22) Filed: Jul. 11, 2019

Related U.S. Application Data
(60) Provisional application No. 62/696,856, filed on Jul. 12, 2018.

(51) Int. Cl. A61F 2/90; A61F 2/07; A61F 2/958

(52) U.S. Cl. CPC A61F 2/90; A61F 2240/001; A61F 2/958; A61F 2/07

(57) ABSTRACT

A stent assembly comprises a stent formed by a network of struts, the stent having an internal surface and an external surface; and a porous fabric material covering at least a portion of at least one of the internal surface and the external surface, and bonded to the stent by a polymer binder that mediates between the struts and the fabric material to bind therebetween. At least 90% of the combined lengths of the struts in a given region are coated with the polymer binder; at least 70%, by area, of fabric disposed in a given region and up to 0.5 mm from a nearest respective strut, is rendered non-porous by the polymer binder within pores of the fabric; at least 70%, by area, of fabric disposed in a given region at least 3 mm from a nearest strut, has pores free of the polymer binder.

### CROSS-REFERENCE TO RELATED APPLICATIONS

This patent application claims the benefit of U.S. Provisional Patent Application No. 62/696,856 filed on Jul. 12, 2018, which is incorporated herein by reference in its entirety.

### FIELD OF THE INVENTION

The present invention relates to medical stent devices and assemblies comprising metal stents and fabric materials attached thereto, along with methods for their manufacture and assembly. In particular, the present invention is suitable for use in medical applications as a sutureless stent graft.

### BACKGROUND

A stent is a metal or polymer tube inserted into the lumen of an anatomic vessel or duct to keep the passageway open. For example, stents may be used in the vascular system, urogenital tract and bile duct, as well as in a variety of other applications in the body. Endovascular stents have become widely used for the treatment of stenosis, strictures, and aneurysms in various blood vessels. These devices are implanted within the vessel to open and/or reinforce collapsing or partially occluded sections of the vessel.

Stents are generally open ended and are radially expandable between a generally unexpanded insertion diameter and an expanded implantation diameter which is greater than the unexpanded insertion diameter. Stents are often flexible in configuration, which allows them to be inserted through and conform to tortuous pathways in the blood vessel. The stent is generally inserted in a radially compressed state and expanded either through a self-expanding mechanism, or through the use of balloon catheters.

It is also known to combine a stent and a graft to form a composite medical device. Grafts are tubular devices which may be formed of a variety of material, including textile and non-textile fabric materials. Such a composite medical device provides additional support for blood flow through weakened sections of a blood vessel. In endovascular applications, the use of a stent/graft combination is becoming increasingly important because the combination not only effectively allows the passage of blood therethrough, but also ensures the implant will remain open and stable. However, the fabric material of the graft is connected to the stent structure by means of stitching or sewing operation. This operation could reduce the overall longitudinal flexibility of the composite device. Longitudinal flexibility is of particular importance to such stent/graft endoluminal prosthesis as the device must be intraluminally delivered through tortuous pathways of a blood vessel to the implantation site. In some cases, an inner sleeve and an outer sleeve are heat-bonded to each other through the open areas between the struts of the stent.

The difficulty encountered in connecting or attaching the fabric material to the stent, is obtaining a proper bond between the stent, which is usually metallic or other material dissimilar to the fabric material. Henceforth, the materials and methods used for attaching fabric material to the stent plays an important role. For example, using polytetrafluoroethylene, only few material bonds well to the polytetrafluoroethylene due to its chemical inert characteristics. The surface of polytetrafluoroethylene material is difficult to wet and the relatively small pore sizes are difficult to penetrate effectively to obtain good mechanical bonds.

Henceforth, there is a need for a method for facilitating ease in attachment of a fabric material or a membrane to the medical stent. Further, there is a need for an option for connecting the fabric or non-fabric material by a simple technique.

### SUMMARY

The present invention discloses methods for facilitating ease in attachment of fabric material to a medical stent. In an embodiment of the present invention, a method for facilitating ease in attachment of fabric material to a medical stent, is disclosed. The method comprises the step of, coating a surface of one or more struts of the stent with a polymer with pre-determined functional group, thereby forming a polymer film layer on the strut of the stent, coating the surface of the strut up to 90%, wherein the surface of the struts is coated by polymer in a circular fashion with at least 30% of the strut wall thickness, and wherein each sides or one or more sides of the surface of the struts further includes a polymer wings ranges from 1 mm to 3 mm, and attaching a woven or a non-woven fabric to the strut of the stent by applying heat.

In one embodiment of the present invention, another method for facilitating ease in attachment of fabric material to a medical stent, is disclosed. This method is done by solvent bonding technique. The method comprises the step of, coating a surface of one or more struts of the stent with a polymer with functional group, thereby forming a polymer film layer on the strut of the stent, coating the surface of the strut up to 90%, wherein the surface of the struts is coated by polymer in a circular fashion with at least 30% of the strut wall thickness, and wherein each sides or one or more sides of the surface of the struts further includes a polymer wings ranges from 1 mm to 3 mm, spraying a solvent on the strut of the stent, attaching a woven or a non-woven fabric to the strut of the stent, and drying the fabric attached stent in an oven for a predetermined time.

In one embodiment, the strut of the stent is encapsulated with the polymer and attaching the fabric material by the application of heat and pressure. In some embodiments, the polymer is melted and flows into a plurality of porous characteristics of the fabric enabling a strong bond between the fabric material and stent. In one embodiment, the melting point of fabric is greater than the melting point of the polymer by at least 10° C. In some embodiments, the fabric material is an electro spun fiber and a perforated thermoplastic polyurethane (TPU) film. In some embodiments, the polymer film has a width of 1-3 mm for each strut on both sides. The polymer film has a wall thickness with 20-80 microns, or 5-70 microns, or 10-40 microns.

According to embodiments, a stent assembly, comprises: (a) a stent formed by a network of struts, the stent having an internal surface and an external surface; and (b) a porous fabric material covering at least a portion of at least one of the internal surface and the external surface, and bonded to the stent by a polymer binder that mediates between the struts and the fabric material to bind therebetween, wherein: (i) at least 90%, by length, of the combined lengths of the struts of the network within a first surface region of the stent, is coated with the polymer binder, (ii) at least 70%, by area, of the fabric material disposed (A) within each one of one or more stent-area portions within a second surface region of the stent and (B) no more than 0.5 mm from a nearest respective strut, is rendered non-porous by a presence of the polymer binder within pores of the fabric material, (iii) at least 70%, by area, of portions of the fabric material (A) disposed within each one of said one or more stent-area portions and within said second surface region and (B) distanced at least 3 mm from a nearest respective strut, is characterized by pores that are free of the polymer binder, and (iv) the thickness of the polymer binder is not less than 1 micron and not greater than 70 microns.

In some embodiments, it can be that (i) the network of struts comprises a plurality of strut segments defined by intersection locations, (ii) an n-sided cell comprises n strut segments defined by n intersection locations, where n is an integer equal to at least 3 and at most 6, and (iii) the first surface region includes at least one n-sided cell. In some such embodiments, the first surface region can include at least 10 n-sided cells.

In some embodiments, it can be that (i) the network of struts includes a plurality of undulating rings of strut segments defined by bends and (ii) the first surface region includes at least one of the undulating rings. In some such embodiments, the first surface region can include at least 5 of the undulating rings.

In some embodiments, the first surface region can include at least 90% of the surface area of the stent.

In some embodiments, it can be that (i) the network of struts comprises a plurality of strut segments defined by intersection locations, (ii) the stent-area portions comprise n-sided cells each comprising n strut segments defined by n intersection locations, where n is an integer equal to at least 3 and at most 6, and (iii) the second surface region includes at least one stent-area portion. In some such embodiments, the second surface region can include at least 10 stent-area portions.

In some embodiments, it can be that (i) the network of struts includes is characterized by undulating rings of strut segments defined by bends, (ii) the stent-area portions comprise undulating rings, and (iii) the second surface region includes at least one stent-area portion. In some such embodiments, the second surface region can include at least 5 stent-area portions.

In some embodiments, the second surface region can include at least 90% of the surface area of the stent.

In some embodiments, the first and second surface regions can be substantially the same surface region.

In some embodiments, the polymer binder coating can be painted onto the struts.

In some embodiments, at least a portion of the struts can be surface-treated before the painting. In some such embodiments, the surface treatment can include a mechanical treatment to increase the surface area of the treated surface. In some embodiments, the surface of the fabric material can receive a surface treatment to improve bonding between struts and fabric.

In some embodiments, it can be that the thickness of the polymer binder is not less than 5 mm and not greater than 40 mm.

In some embodiments, it can be that at least 70%, by area, of the fabric material disposed (A) within each one of one or more stent-area portions within a second surface region of the stent and (B) no more than 1 mm from a nearest respective strut, are rendered non-porous by a presence of the polymer binder within pores of the fabric material.

In some embodiments, it can be that at least 70%, by area, of the fabric material disposed (A) within each one of one or more stent-area portions within a second surface region of the stent and (B) no more than 2 mm from a nearest respective strut, are rendered non-porous by a presence of the polymer binder within pores of the fabric material.

In some embodiments, it can be that at least 70%, by area, of portions of the fabric material (A) disposed within each one of said one or more stent-area portions and within said second surface region and (B) distanced at least 2 mm from a nearest respective strut, is characterized by pores that are free of the polymer binder.

In some embodiments, it can be that at least 70%, by area, of portions of the fabric material (A) disposed within each one of said one or more stent-area portions and within said second surface region and (B) distanced at least 3 mm from a nearest respective strut, is characterized by pores that are free of the polymer binder.

In some embodiments, it can be that (i) the binder coating forms a pair of binder-coating wings extending laterally in respective opposite directions from each of a plurality of the binder-coated struts, and (ii) the bonding of the fabric to the surface of the stent includes bonding the fabric to at least part of each binder wing.

A method is disclosed, according to embodiments, for attaching a fabric material to a stent comprising a metal alloy, the stent formed by a network of struts and having an external surface and an internal surface. The method comprises: (a) engaging a porous fabric material with at least some of the surfaces of the struts; and (b) applying a polymer binder so as to bond the porous fabric material with said at least some of the surfaces of the struts, the applying being such that: (i) at least 90%, by length, of the combined lengths of the struts of the network within a first surface region of the stent, are bonded to the porous fabric material by polymer binder, (ii) at least 70%, by area, of the fabric material disposed (A) within each one of one or more stent-area portions within a second surface region of the stent and (B) no more than 0.5 mm from a nearest respective strut, is rendered non-porous by a presence of the polymer binder within pores of the fabric material, (iii) at least 70%, by area, of portions of the fabric material (A) disposed within each one of said one or more stent-area portions and within said second surface region and (B) distanced at least 3 mm from a nearest respective strut, is characterized by pores that are free of the polymer binder, and (iv) the thickness of the polymer binder is not less than 1 micron and not greater than 70 microns.

In some embodiments, it can be that (i) the network of struts comprises a plurality of strut segments defined by intersection locations, (ii) an n-sided cell comprises n strut segments defined by n intersection locations, where n is an integer equal to at least 3 and at most 6, and (iii) the first surface region includes at least one n-sided cell. In some such embodiments, the first surface region can include at least 10 n-sided cells.

In some embodiments, it can be that (i) the network of struts includes a plurality of undulating rings of strut segments defined by bends and (ii) the first surface region includes at least one of the undulating rings. In some such embodiments, the first surface region can include at least 5 of the undulating rings.

In some embodiments, the first surface region can include at least 90% of the surface area of the stent.

In some embodiments, it can be that (i) the network of struts comprises a plurality of strut segments defined by intersection locations, (ii) the stent-area portions comprise n-sided cells each comprising n strut segments defined by n intersection locations, where n is an integer equal to at least 3 and at most 6, and (iii) the second surface region includes at least one stent-area portion. In some such embodiments, the second surface region can include at least 10 stent-area portions.

In some embodiments, it can be that (i) the network of struts is characterized by undulating rings of strut segments defined by bends, (ii) the stent-area portions comprise undulating rings, and (iii) the second surface region includes at least one stent-area portion. In some such embodiments, the second surface region can include at least 5 stent-area portions.

In some embodiments, the second surface region can include at least 90% of the surface area of the stent.

In some embodiments, the first and second surface regions can be the same surface region.

In some embodiments, the applying of the polymer binder coating can include painting.

In some embodiments, the method can additionally include, prior to the applying, treating a portion of the surface of the stent, wherein the applying is to at least a portion of the surface-treated part of the stent. In some such embodiments, the surface treatment can include a mechanical treatment to increase the surface area of the treated surface.

In some embodiments, the applying can be such that the thickness of the polymer binder is not less than 5 mm and not greater than 40 mm.

In some embodiments, the applying can be such that at least 70%, by area, of the fabric material disposed (A) within each one of one or more stent-area portions within a second surface region of the stent and (B) no more than 1 mm from a nearest respective strut, are rendered non-porous by a presence of the polymer binder within pores of the fabric material.

In some embodiments, the applying can be such that at least 70%, by area, of portions of the fabric material (A) disposed within each one of said one or more stent-area portions and within said second surface region and (B) distanced at least 2 mm from a nearest respective strut, is characterized by pores that are free of the polymer binder.

In some embodiments, the applying can be such that at least 70%, by area, of portions of the fabric material (A) disposed within each one of said one or more stent-area portions and within said second surface region and (B) distanced at least 2 mm from a nearest respective strut, is characterized by pores that are free of the polymer binder.

In some embodiments, the applying can include forming a pair of binder-coating wings extending laterally in respective opposite directions from each of a plurality of the binder-coated struts, and (ii) the bonding of the fabric to the surface of the stent includes bonding the fabric to at least part of each binder wing.

According to embodiments, a stent assembly can be manufactured according to any one of the methods disclosed hereinabove.

According to embodiments, a radially compressible covered stent assembly comprises first and second stent-assembly segments displaced from one another longitudinally, the stent assembly having an external surface and an internal surface. The stent comprises: (a) a radially compressible stent formed by a network of struts, the network having a plurality of intersection locations at which intersections and/or bends define strut segments, each strut segment having respective outward-facing and inward facing-surfaces which correspond to the external and internal surfaces of the stent assembly, and two respective laterally-facing surfaces; (b) a polymer-binder coating applied to the struts so as to at least partially coat at least some of the strut segments and at least some of the intersection locations; and (c) a fabric covering at least a portion of at least one of the internal surface and the external surface so as to be in contact with the polymer-binder coating, at least 80% of said contact being characterized by the polymer-binder coating forming a bond between the fabric and a strut segment or intersection location at the respective point of contact, wherein (i) first and second stent-assembly segments are at least partially coated with the polymer-binder coating with respective first and second coating thicknesses on one or both laterally-facing surfaces of at least some respective strut segments at or adjacent to respective intersection locations, said first and second coating thicknesses being different from each other, and (ii) the radial strength of the first stent-assembly segment is at least 20% greater than the radial strength of the second stent-assembly segment.

In some embodiments, the first and second stent-assembly segments can be contiguous to one another.

In some embodiments, the first and second stent-assembly segments can be annular.

In some embodiments, the struts can comprise a metal or a metal alloy.

In some embodiments, the struts can comprise nitinol.

In some embodiments, it can be that over at least a portion of the stent, the network is characterized by n-sided cells comprising n strut segments defined by n intersections, where n is an integer equal to no less then 3 and no more than 6.

In some embodiments, it can be that over at least a portion of the stent, the network is characterized by undulating rings of strut segments defined by bends.

In some embodiments, the fabric can be a non-woven, impermeable fabric.

In some embodiments, the fabric can be porous.

In some embodiments, the polymer-binder coating can comprise a thermoplastic elastomer.

In some embodiments, the first and second coating thicknesses can be both the same one of an average thickness, a minimum thickness and a maximum thickness.

In some embodiments, the radial strength of the first stent-assembly segment can be at least 50% greater than the radial strength of the second stent-assembly segment.

In some embodiments, the radial strength of the first stent-assembly segment can be at least 80% greater than the radial strength of the second stent-assembly segment.

A method is disclosed, according to embodiments, for producing a radially compressible stent assembly comprising longitudinally displaced stent-assembly segments with different respective radial strengths. The method comprises: (a) applying a polymer-binder coating to at least a portion of a radially compressible stent formed by a network of struts having a plurality of intersection locations at which intersections and/or bends define strut segments, each strut segment having respective outward-facing and inward facing-surfaces which correspond to the external and internal surfaces of the stent assembly, and two respective laterally-facing surfaces; (b) bonding a stent-covering fabric to at least some of the strut segments and intersection locations, at an elevated temperature above the melting temperature of the polymer-binder coating and below the melting temperature of the fabric, the polymer-binder coating when cooled forming a bond between the fabric and respective strut segments and intersection locations, wherein the applying includes applying a first coating thickness on one or both laterally-facing surfaces of at least some respective strut segments at or adjacent to respective intersection locations of a first stent-assembly segment and applying a second coating thickness on one or both laterally-facing surfaces of at least some respective strut segments at or adjacent to respective intersection locations of a second stent-assembly segment, and the respective stent-assembly segments having different radial strengths that are a function of the coating thicknesses.

In some embodiments, the applying can include painting.

In some embodiments, the method of can additionally include, prior to the applying, treating the surface of at least part of the stent, wherein the applying is to at least a portion of the surface-treated part of the stent. In some such embodiments, the surface treatment can includes a mechanical treatment to increase the surface area of the treated surface.

In some embodiments, the first and second stent-assembly segments can be contiguous to one another.

In some embodiments, the struts can comprise nitinol.

In some embodiments, it can be that wherein over at least a portion of the stent, the network is characterized by n-sided cells comprising n strut segments defined by n intersections, where n is an integer equal to no less then 3 and no more than 6.

In some embodiments, the fabric can be a non-woven, impermeable fabric.

In some embodiments, the fabric can be porous.

In some embodiments, the polymer-binder coating can comprise a thermoplastic elastomer.

In some embodiments, the first and second coating thicknesses can both be the same one of an average thickness, a minimum thickness and a maximum thickness.

In some embodiments, the radial strength of the first stent-assembly segment can be at least 50% greater than the radial strength of the second stent-assembly segment.

In some embodiments, the radial strength of the first stent-assembly segment can be at least 80% greater than the radial strength of the second stent-assembly segment.

According to embodiments, a stent assembly can be manufactured according to any one of the methods disclosed hereinabove.

According to embodiments, a stent assembly comprises: (a) a metal stent formed by a network of struts and having an internal major surface and an external major surface; (b) a binder coating comprising a polymer and covering at least a portion of at least some of the struts, and (c) a non-porous fabric at least partly covering a single one of the two major surfaces and bonded thereto by the binder coating, wherein (i) the binder coating forms a pair of binder-coating wings extending laterally in respective opposite directions from each of a plurality of the binder-coated struts, and (ii) the bonding of the fabric to the surface of the stent includes bonding the fabric to at least part of each binder wing.

In some embodiments, the polymer can comprise a thermoplastic elastomer.

In some embodiments, the non-porous fabric can comprise a liquid-impermeable film.

In some embodiments, the non-porous fabric can comprise a thermoplastic elastomer.

In some embodiments, the polymer and the non-porous fabric can both comprise the same thermoplastic elastomer.

In some embodiments, it can be that each wing has a minimum width, as measured from an edge of the wing to an edge of the metal substrate of the corresponding binder-coated strut, of at least 1 mm. In some such embodiments, the minimum width can be at least 2 mm. In some such embodiments, the minimum width can be at least 3 mm.

A method is disclosed, according to embodiments, for producing a stent assembly comprising (i) a metal stent formed by a network of struts and having an internal major surface and an external major surface, and (ii) a liquid-impermeable fabric at least partly covering a single one of the two major surfaces. The method comprising: (a) surface-treating at least some of the surfaces of the struts; (b) applying a binder coating comprising a polymer to at least a portion of at least some of the surface-treated struts; and (c) bonding a non-porous fabric to at least some of the binder-coated surface-treated struts on either the internal major surface or the external major surface of the metal stent, wherein (i) the applying includes forming a pair of binder-coating wings extending laterally in respective opposite directions from a binder-coated surface-treated strut, said opposite directions being locally parallel to one of the two major surfaces of the metal stent, and (ii) the bonding includes bonding the fabric to at least part of each binder wing.

In some embodiments, the polymer can comprise a thermoplastic elastomer.

In some embodiments, the non-porous fabric can comprise a liquid-impermeable film.

In some embodiments, the non-porous fabric can comprise a thermoplastic elastomer.

In some embodiments, the polymer and the non-porous fabric can both comprise the same thermoplastic elastomer.

In some embodiments, the applying of the binder coating can include painting.

In some embodiments, the surface-treating can include performing a mechanical treatment to increase the surface area of the treated surface.

In some embodiments, it can be that each wing has a minimum width, as measured from an edge of the wing to an edge of the metal substrate of the corresponding binder-coated strut, of at least 1 mm.

In some embodiments, it can be that each wing has a maximum width, as measured from an edge of the wing to an edge of the metal substrate of the corresponding binder-coated strut, of at most 3 mm.

According to embodiments, a stent assembly can be manufactured according to any of the methods disclosed hereinabovein claims 78 to 86.

### BRIEF DESCRIPTION OF THE DRAWINGS

The invention will now be described further, by way of example, with reference to the accompanying drawings, in which the dimensions of components and features shown in the figures are chosen for convenience and clarity of presentation and not necessarily to scale. In the drawings:

FIGS. 1A and 1B illustrate a stent characterized by n-sided cells, according to embodiments of the present invention.

FIGS. 2A and 2B illustrate a stent characterized by undulating rings, according to embodiments of the present invention.

FIGS. 3 and 4 illustrate stent designs with different types of intersection locations, according to embodiments of the present invention.

FIG. 5 shows a perspective view of a stent coated with a polymer binder, according to embodiments of the present invention.

FIG. 6 shows a perspective view of a stent assembly according to embodiments of the present invention.

FIGS. 7A and 7B are schematic cross-sectional illustrations of bonding a fabric to a stent by having a polymer binder enter pores in the fabric, according to embodiments of the present invention;

FIG. 8 shows a flowchart of a method for attaching a fabric material to a stent comprising a metal alloy, according to embodiments of the present invention.

FIGS. 9A and 9B illustrate a stent assembly characterized by stent-assembly segments, according to embodiments of the present invention.

FIGS. 10A and 10B are schematic illustrations of intersecting struts coated with a polymer binder, according to embodiments of the present invention.

FIG. 11 shows a flowchart of a method for producing a radially compressible stent assembly comprising longitudinally displaced stent-assembly segments with different respective radial strengths, according to embodiments of the present invention.

FIGS. 12A and 12B are schematic cross-sectional illustrations of binder wings provided to facilitate bonding of a fabric to a stent, according to embodiments of the present invention.

FIG. 13 shows a flowchart of a method a method for producing a stent assembly comprising a metal stent formed by a network of struts and having an internal major surface and an external major surface, according to embodiments of the present invention.

FIG. 14 illustrates the painting of a binder onto a stent with fabric engaged therewith, according to embodiments of the present invention.

FIGS. 15A and 15B illustrate examples of stent designs in which a fabric material can be attached to portions of stents, according to embodiments of the present invention.

FIGS. 16A-16E illustrate examples of different shapes and designs of stents incorporating embodiments of the present invention.

### DETAILED DESCRIPTION OF THE ILLUSTRATED EMBODIMENTS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice. Throughout the drawings, like-referenced characters are generally used to designate like elements. Subscripted reference numbers (e.g., 10₁) or letter-modified reference numbers (e.g., 100a) are used to designate multiple separate appearances of elements in a single drawing, e.g. 10₁ is a single appearance (out of a plurality of appearances) of element 10, and 100a is a single appearance (out of a plurality of appearances) of element 100.

For convenience, in the context of the description herein, various terms are presented here. To the extent that definitions are provided, explicitly or implicitly, here or elsewhere in this application, such definitions are understood to be consistent with the usage of the defined terms by those of skill in the pertinent art(s). Furthermore, such definitions are to be construed in the broadest possible sense consistent with such usage.

The term ‘stent assembly’ as used herein means an assembly of a medical stent and a fabric cover, sleeve or attachment, attached to the struts of the stent. A stent assembly can be what is commonly called a stent graft. A stent assembly can additionally include materials used in the assembly, such as, for example, primers, coatings, binders and polymers or elastomers.

The term ‘bonding’ can be used to mean a process of joining using any one or more of: applying an adhesive binder, e.g., by painting it onto a surface of a stent strut; applying heat; and applying pressure. The bonding can be carried out by applying a binder to a strut and then engaging the fabric, or by engaging the fabric and then applying the binder. Either approach can be practiced in any of the embodiments disclosed herein.

In some embodiments, a stent assembly comprises a stent and a porous fabric material. In other embodiments, a stent assembly comprises a stent and a non-porous fabric, which can be liquid-impermeable. The fabric can be in the form of a sheet or a sleeve, e.g., a cylinder.

### First Discussion of Embodiments

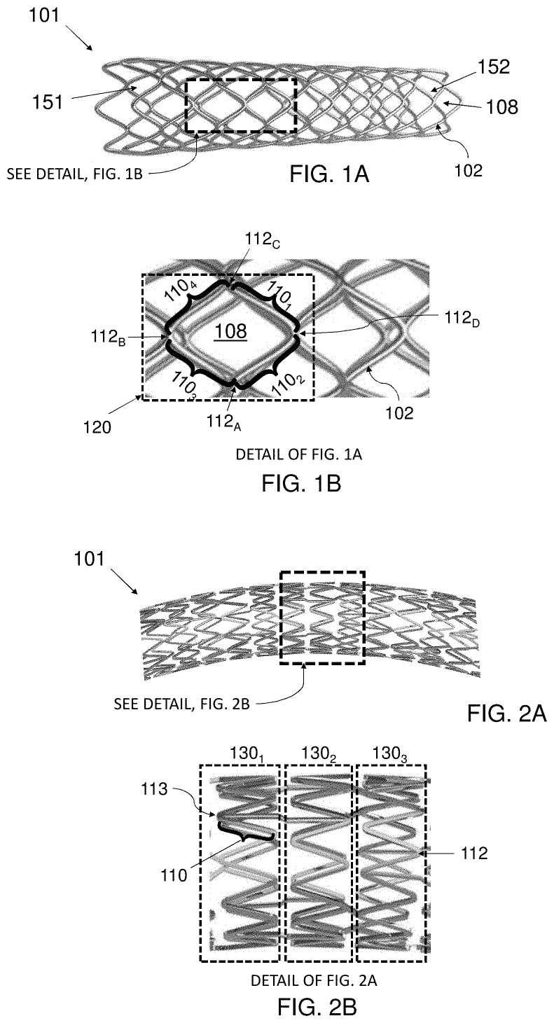

A first example of a stent is shown in FIGS. 1A and 1B. The stent 101 is formed by a network of struts 102. The stent 101 has an internal surface 151 and an external surface 152. As seen, each of the surfaces 151, 152 comprises surfaces of struts 102, and open spaces 108 between the struts 102. The network of struts 102 can comprise a plurality of strut segments 110 defined by intersection locations 112. The area of either surface 151, 152 of the stent 102 can be thought of as comprising a plurality of stent-area portions.

In the embodiment illustrated in FIGS. 1A and 1B, the stent-area portions comprise 4-sided cells 120 each comprising 4 strut segments (e.g., 110₁, 110₂, 110₃, 110₄) defined by 4 intersection locations (e.g., 112_{A}, 112_{B}, 112_{C}, 112_{D}). More generally, the stent-area portions, in embodiments, can comprise n-sided cells each comprising n strut segments defined by n intersection locations, where n is an integer equal to at least 3 and at most 6. In various embodiments, the cells can have either regular polygon shapes or irregular shapes. Strut segments 110 can be mostly straight or curved in accordance with a stent design. A stent 101 can have at least 1, at least 10, at least 20, at least 50, at least 100 or more such n-sided cells. The n-sided cells can make up one or more regions of the surfaces 151, 152 of a stent 101, or can account for the entire stent surface.

Referring now to FIGS. 2A and 2B, another example of stent design is shown where the network of struts 102 is characterized by undulating rings 130 of strut segments 102 defined by bends 113. Bends 113 are another form of intersection locations like the intersection locations 112 of FIG. 1B, in that they mark where strut segments 102 intersect. As mentioned earlier, the stent 101 can be thought of as comprising a plurality of stent-area portions, in the embodiment illustrated in FIGS. 2A and 2B, the stent-area portions comprise undulating rings 130 (e.g., 130₁, 130₂, 130₃). A stent 101 can have at least 1, at least 5, at least 10, at least 20, or more such undulating rings 130. The undulating rings 130 can make up one or more regions of the surfaces 151, 152 of a stent 101, or can account for the entire stent surface.

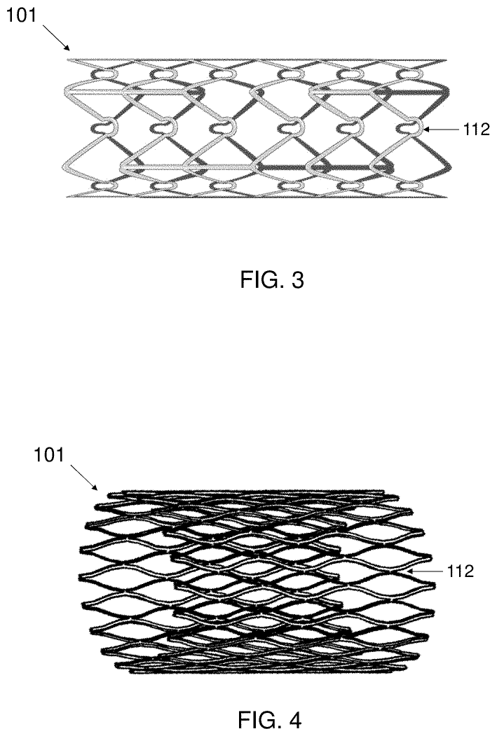

FIGS. 3 and 4 illustrate additional examples of stents 101 characterized by n-sided cells 120, wherein the intersection locations 112 are different from those shown, for example, in FIGS. 1A and 1B. In FIG. 3, the intersection locations 112 comprise overlapping hooks. In FIG. 4, the 4-sided cells 120 are laterally compressed and the constituent strut segments are somewhat curved; the corresponding intersection locations 112 are located where two such adjacent ‘curves’ touch each other. In spite of having a different form than the earlier examples of n-sided cells 120 and intersection locations 112, the actual design is not material to the invention, and any suitable strut design can be used. Similarly, the undulating rings 130 of FIGS. 2A and 2B can be, in embodiments, less regular and/or can have more complex shapes.

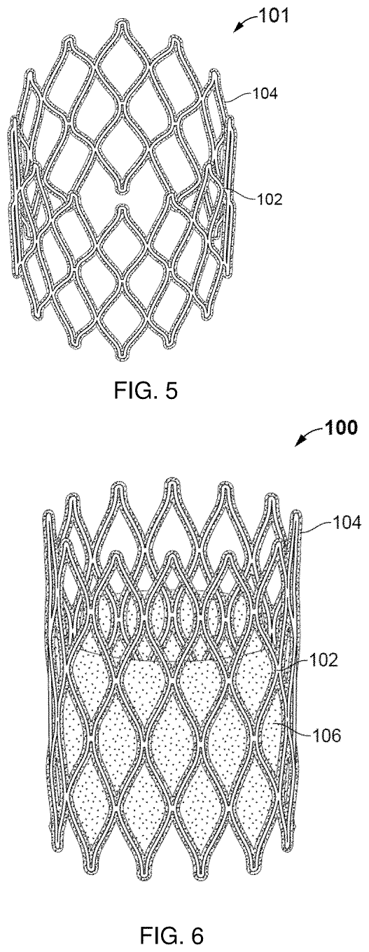

FIG. 5 shows a view of a stent 101 coated with a polymer to form a polymer binder layer 104, according to an embodiment. Generally, the medical stent or stent 101 is a tiny tube, comprising a plurality or network of struts 102 configured to form a mesh like structure. The width of the strut 102 is typically between 1-2 mm, although it can be narrower or wider according to specific stent designs. According to the present invention, surfaces of the struts 102 of the stent 101 can be coated with a polymer binder. In embodiments, the binder layer 104 can comprise a thermoplastic elastomer chosen for its thermoplastic and elastomeric properties. Examples of suitable thermoplastic elastomers include styrenic block copolymers, thermoplastic polyolefinelastomers, thermoplastic vulcanizates, thermoplastic polyurethanes, thermoplastic copolyesters, and thermoplastic polyamides.

We now refer to FIG. 6 which shows a stent assembly 101 comprising a stent 101 with struts 102 coated with a polymer binder 104 and partly covered by a fabric material 106. In one embodiment, the fabric material 106 is attached to struts 102 of by means of a solvent bonding technique. In another embodiment, the fabric material 106 is attached to the struts 102 by means of heat and pressure application. In some embodiments, the fabric material 106 is fabricated from any desired fiber material used in the industry for stent sleeves, covers and grafts, such as, but not limited to, electro spun fibers, expanded polytetrafluoroethylene (EPTFE), or thermoplastic polyurethane (TPU) film. The fabric material 106 can be manufactured by means of any known manufacturing technology, but not limited to woven, non-woven, knitting or electrospinning technique.

In some embodiments, a porous fabric material can be deployed to cover at least a portion of at least one of the internal surface 151 and the external surface 152, and bonded to the metal stent 100 by a polymer binder 104 that mediates between the struts 102 and the fabric material 106 to bind therebetween.

As seen in FIG. 6, in some embodiments, at least 60%, by length, of the combined lengths of the struts 102 of the network within a first surface region of the stent, is coated with the polymer binder 104. In other embodiments, at least 70%, at least 80%, or least 90% of the combined lengths of the struts 102 can be coated with the polymer binder 104.

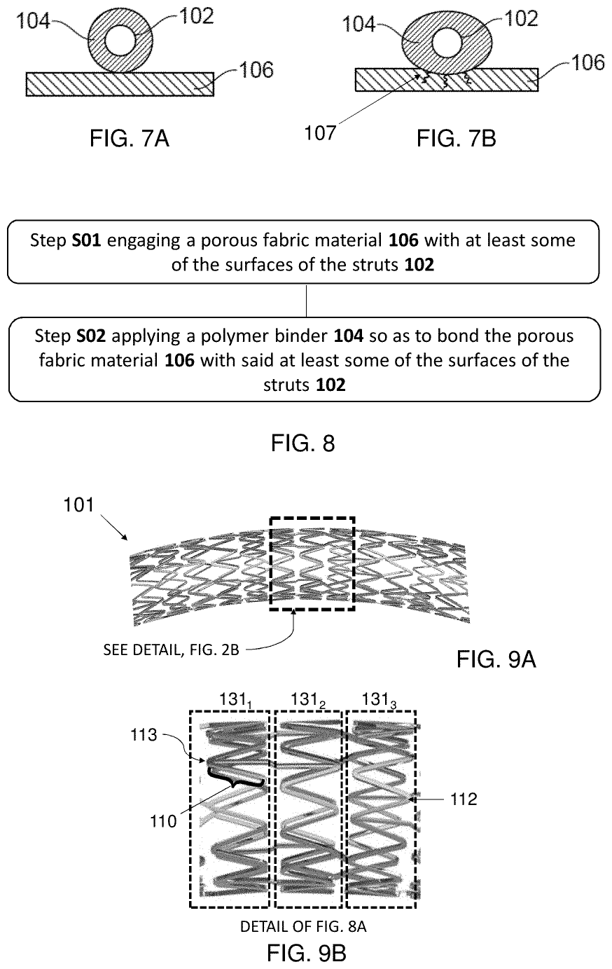

As is illustrated schematically in FIGS. 7A and 7B, it can be desirable for the polymer binder 104 to enter pores 107 in fabric 106 as a way of making the binding/bonding between fabric 106 and more effective. Returning to FIG. 6, the polymer binder 104 is applied (or expands/flows/is squeezed by pressure) close to the struts 102 so as to enter pores 107 in the fabric 106 and is not to be found far from the struts 102. In embodiments, for a given region (or multiple regions) comprising one or more stent-area portions, at least 70% or at least 80% of the area of the fabric material 106 that is “close to struts 102” is rendered non-porous (meaning at least 90% non-porous) by a presence of the polymer binder 104 within pores 107 of the fabric material 106. “Close to struts 102” can be interpreted as with 0.5 mm, or within 1.0 mm, or with 2.0 mm, where the distance is measured laterally from lateral edges of the struts 102. Similarly, in those region(s), at least 70% or at least 80% of the area the fabric 106 in of portions of the fabric material that is “far from struts 102” is characterized by pores 107 that are free (meaning at least 90% free) of the polymer binder 104. “Far from struts 102” can be interpreted as at least 1 mm, at least 2 mm, or at least 3 mm displaced laterally from the lateral edges of struts 102.

It should be noted that any of the foregoing criteria (e.g., with respect to at least 70% or at least 80% of the area of the fabric material 106 that is “close to struts 102” being rendered non-porous, or with respect to at least 70% or at least 80% of the area the fabric 106 in of portions of the fabric material that is “far from struts 102” being characterized by pores 107 that are free of the polymer binder 104) can be applied globally for all stent-areas (e.g., n-sided cells 110 and/or undulating rings 130) in a region of the stent or even over the entire stent, but can also be applied at the individual stent-area (n-sided cell 110 and/or undulating ring 130) level, such that in some embodiments the criteria are applied within each individual one of the stent-areas.

According to some embodiments, the thickness of the polymer binder 104 is not less than 1 micron and not greater than 70 microns. In some embodiments, the thickness of the polymer binder 104 is not less than 5 microns and not greater than 40 microns.

In embodiments, the struts 102 are encapsulated with the polymer 104 and the fabric material 106 is attached thereto by the application of heat and pressure. In some embodiments, the fabric 106 is engaged (i.e., brought in contact) with the bare metal strut 102 and the polymer binder 104 is then applied so as to bind therebetween. In some embodiments, the polymer 104 is melted and flows into a plurality of pores 107 characteristics of the fabric 106 enabling a strong bond between the fabric material 106 and struts 102. In some embodiments, the melting point of fabric 106 is greater than the melting point of the polymer by at least 10° C.

The flowchart in FIG. 8 illustrates a method for attaching a fabric material 106 to a stent 101 comprising a metal alloy, where the stent 100 is formed by a network of struts 102 and having an external surface 151 and an internal surface 152. The method comprises:

Step S01 engaging a porous fabric material 106 with at least some of the surfaces of the struts 102; and

Step S02 applying a polymer binder 104 so as to bond the porous fabric material 106 with said at least some of the surfaces of the struts 102. The applying is such that: (i) at least 90%, by length, of the combined lengths of the struts of the network within a first surface region of the stent, are bonded to the porous fabric material by polymer binder, (ii) at least 70%, by area, of the fabric material disposed (A) within each one of one or more stent-area portions within a second surface region of the stent and (B) no more than 2 mm from a nearest respective strut, is rendered non-porous by a presence of the polymer binder within pores of the fabric material, (iii) at least 70%, by area, of portions of the fabric material (A) disposed within each one of said one or more stent-area portions and within said second surface region and (B) distanced at least 3 mm from a nearest respective strut, is characterized by pores that are free of the polymer binder, and (iv) the thickness of the polymer binder is not less than 1 micron and not greater than 70 microns.

In some embodiments, the fabric 106 can receive surface treatment, additionally or alternatively to the surface treatment of struts 102, so as to improve the bonding of the fabric with the struts.

### Second Discussion of Embodiments

Precise application of polymer binder to struts can be desirable because it facilitates control of physical parameters of a stent or stent assembly, such as, for example, radial strength (also called radial resistance). In embodiments, radial strength (a measure of stiffness of a segment of a stent can be a function of the lateral thickness of the polymer binder coating at or adjacent to intersection locations of struts, assuming all else (material, strut thickness) is held constant.

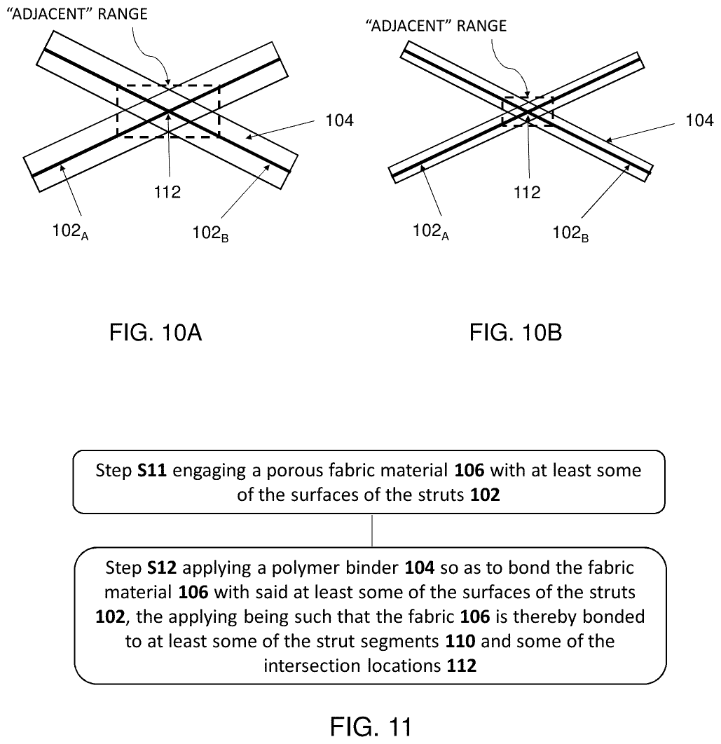

With precise control of radial strength, it is possible to deploy a stent with different radial strengths in different segments. Referring now to FIGS. 9A and 9B (similar to FIGS. 2A and 2B, except that in FIGS. 9A and 9B the undulating rings 130 of the earlier figures are used to embody stent segments 131), in an embodiment, stent segment 131₂ can have a radial strength that is higher (e.g., at least 20% higher, at least 50% higher, or at least 80% higher) than stent segments 131₁ and 131₃, depending on the thickness of a polymer binding (not shown in FIGS. 2A and 2B) applied at or adjacent to intersection locations 112. Such segments are obviously not limited to stents characterized by the undulating rings of FIGS. 2A and 2B and can alternatively or additionally include stent surface regions characterized by n-sided cells. The term “adjacent” is used to mean within an “adjacent range” where the polymer binder 104 of one strut 102 intersects with the polymer binder 104 of another strut 102 at an intersection location 112, as illustrated schematically FIGS. 10A and 10B.

In FIGS. 10A and 10B, it can be seen that the polymer binder 104 can be applied with different lateral thicknesses depending, inter alia, on the desired radial strength, i.e., although the figures accompanying this specification are not drawn to scale, the thickness of polymer binder 104 in FIG. 10A has been deliberately and exaggeratedly shown to be much thicker than in FIG. 10B. It will be obvious to the skilled artisan that the illustration of FIGS. 10A and 10B is equally applicable to a stent surface region where the intersection locations are characterized by bends (like those in FIGS. 2A and 2B).

In embodiments, a radially compressible covered stent assembly 100 comprises first and second stent-assembly segments 131 displaced from one another longitudinally, the stent assembly 100 has an external surface 152 and an internal surface 151, and the stent assembly comprises: (a) a radially compressible stent 101 formed by a network of struts 102, the network having a plurality of intersection locations 112 at which intersections and/or bends define strut segments 110, each strut segment 110 having respective outward-facing and inward facing-surfaces which correspond to the external and internal surfaces of the stent assembly 152, 151, and two respective laterally-facing surfaces; (b) a polymer binder 104 applied to the struts 102 so as to at least partially coat at least some of the strut segments 110 and at least some of the intersection locations 112; and a fabric 106 covering at least a portion of at least one of the internal surface 151 and the external surface 152 so as to be in contact with the polymer binder 104, where at least 80% of said contact is characterized by the polymer binder 104 forming a bond between the fabric 106 and a strut segment 110 or intersection location 112 at the respective point of contact. First and second stent-assembly segments 131 are at least partially coated with the polymer binder 104, the polymer binder 104 having respective first and second thicknesses on one or both laterally-facing surfaces of at least some respective strut segments 110 at or adjacent to respective intersection locations 112, said first and second thicknesses being different from each other. The radial strength of the first stent-assembly segment 131 is greater than the radial strength of the second stent-assembly segment 131. In some embodiments, the thickness are different from each other by at least 10% and the radial strength of the segments 131 differs by at least 20%.

These embodiments can be beneficially combined with any of the other embodiments disclosed herein, e.g., with respect to the network of struts being characterized by n-sided cells (as in FIGS. 1A and 1B), with respect to the network of struts being characterized by undulating rings (as in FIGS. 2A and 2B), or with respect to the stated ranges of thickness of the polymer binder. In embodiments, the network of struts 102 can comprise a plurality of strut segments 110 defined by intersection locations 112, where an n-sided cell comprises n strut segments 110 defined by n intersection locations 112, where n is an integer equal to at least 3 and at most 6, and at least 70% or at least 80% of the surface area of the metal stent is characterized by n-sided cells. In embodiments, the network of struts 102 includes a plurality of undulating rings 130 of defined by bends, and at least 70% of the surface area or at least 80% of the metal stent 101 is characterized by undulating rings 130. In embodiments, the thickness of the polymer binder 104 is not less than 1 micron and not greater than 70 microns. The flowchart in FIG. 11 illustrates a method for producing a radially compressible stent assembly 100 comprising longitudinally displaced stent-assembly segments 131 with different respective radial strengths, the stent assembly 100 comprising a radially compressible stent 101 formed by a network of struts 102, the network having a plurality of intersection locations 112 at which intersections and/or bends define strut segments 110, each strut segment 110 having respective outward-facing and inward facing-surfaces which correspond to the external and internal surfaces 152, 151 of the stent assembly, and two respective laterally-facing surfaces. The method comprises:

Step S11 engaging a fabric material 106 with at least some of the surfaces of the struts 102; and

Step S12 applying a polymer binder 104 so as to bond the fabric material 106 with said at least some of the surfaces of the struts 102, the applying being such that the fabric 106 is thereby bonded to at least some of the strut segments 102 and some of the intersection locations 112. The applying includes applying a first coating thickness on one or both laterally-facing surfaces of at least some respective strut segments 110 at or adjacent to respective intersection locations 112 of a first stent-assembly segment 131 and applying a second coating thickness on one or both laterally-facing surfaces of at least some respective strut segments 110 at or adjacent to respective intersection locations 112 of a second stent-assembly segment 131; the respective stent-assembly segments 131 have different radial strengths that are a function of the coating thicknesses.

### Third Discussion of Embodiments

According to embodiments, it can be desirable to bond a non-porous fabric material to a stent, i.e., without the benefit of binder filling pores to improve the efficiency of bonding. For example, lateral ‘wings’ of binder material on either side of a stent strut can be provided so as to increase the area of binding contact between the polymer binder and the fabric. The wings are provided laterally, i.e., locally parallel to the surface of the stent without necessarily thickening the coating on the inward-facing or outward-facing surfaces of the struts.

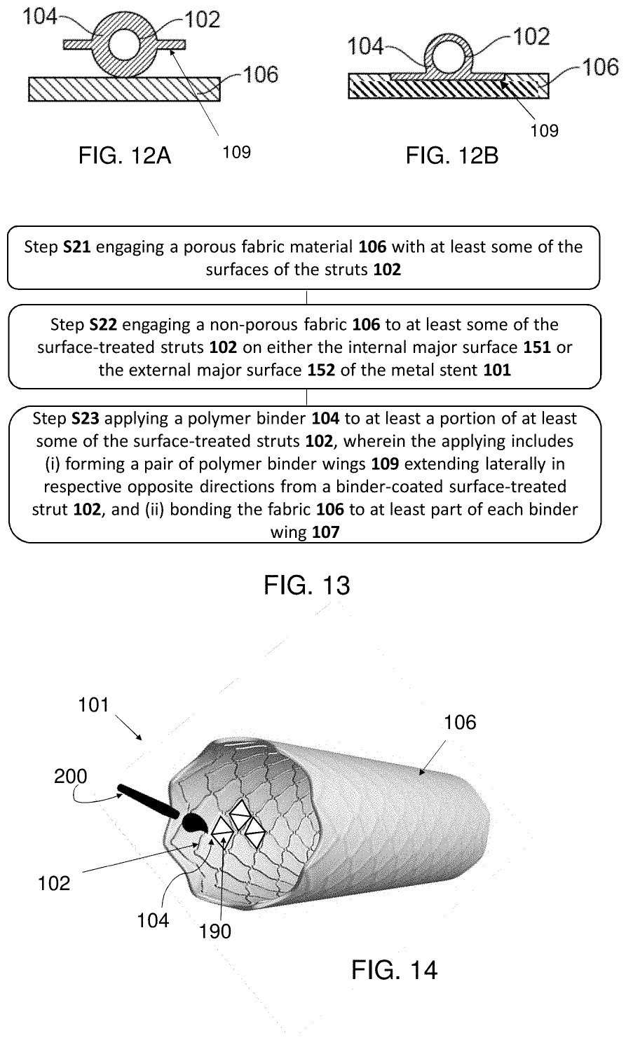

Examples of wings are shown in FIGS. 12A and 12B. In FIG. 12A, wings 109 are formed laterally from strut 102 and extent the polymer coating 104 to both sides of the strut 102. In FIG. 12B, the wings 109 are bonded with a corresponding area of the surface of the fabric 106.

In embodiments, a fabric 106 can cover all, part, or none of one of the surfaces 151, 152 of a stent 101. A coverage-value reflects to what extent a surface is covered. For example, a 100% coverage-value means that a surface is completely covered, a 50% coverage-value means that 50% of the area of a surface is covered, and a 0% coverage-value means that a surface has no fabric cover at all.

In embodiments, a stent assembly 100 comprises: a metal stent 101 formed by a network of struts 102 and having an internal major surface 151 and an external major surface 152; a polymer binder coating 104, covering at least a portion of at least some of the struts 102, and having a thickness not less than 1 micron and not greater than 70 microns (in some embodiments 5-40 microns); and a fabric 106 at least partly covering at least one of the two major surfaces 151, 152 and bonded thereto by the binder coating 104, such that a first surface (i.e., internal or external surface 151 or 152) has a coverage-value of no less than 50%, and the second surface (i.e., the other of the two surfaces 151, 152) has a coverage-value of at most 50% of the coverage-value of the first surface. In an example, a first surface has a coverage-value of over 90% and the second surface has a coverage-value of 0%. In another example, a first surface has a coverage-value of 50% and the second surface has a coverage value of 10%. Further, the binder coating 104 forms a pair of binder-coating wings 109 extending laterally in respective opposite directions from each of a plurality of the binder-coated struts 102, and the bonding of the fabric 106 to the surface of the stent 101 includes bonding the fabric 106 to at least part of each binder wing 109. In some embodiments, the coverage-value of the second surface is zero. In some embodiments, the fabric 106 can be a non-porous, liquid impermeable film.

The flowchart in FIG. 13 illustrates a method for producing a stent assembly 100 comprising a metal stent 101 formed by a network of struts 102 and having an internal major surface 151 and an external major surface 152, and (ii) a liquid-impermeable fabric 106 at least partly covering a single one of the two major surfaces 151, 152. The method comprises:

Step S21 surface-treating at least some of the surfaces of the struts 102; Surface treatment are known in the art and can be mechanical (e.g., sandblasting, creating pits or striations, etc., to increase surface area), or chemical (e.g., etching, eroding, dipping, etc.).

Step S22 engaging a non-porous fabric 106 to at least some of the surface-treated struts 102 on either the internal major surface 151 or the external major surface 152 of the metal stent 101; and

Step S23 applying a polymer binder 104 to at least a portion of at least some of the surface-treated struts 102, wherein the applying includes (i) forming a pair of polymer binder wings 109 extending laterally in respective opposite directions from a binder-coated surface-treated strut 102, and (ii) bonding the fabric 106 to at least part of each binder wing 107. In some embodiments, the applying includes painting.

In some embodiments, the fabric 106 can receive surface treatment, additionally or alternatively to the surface treatment of struts 102, so as to improve the bonding of the fabric with the struts.

FIG. 14 shows a metal stent, with a fabric material 106 engaged on the external surface of the stent. The example shown in FIG. 14 is of a stent with fabric only on the external surface, but it will obvious to the skilled practitioner that the teaching herein applies equally to a stent with fabric on the internal surface and to stents fabric on both major surfaces, i.e., internal and external. According to embodiments, a polymer binder 104 is applied to the struts 102. In some embodiments, the application is by painting the binder onto the struts, as indicated schematically by paintbrush 200. In some embodiments, sections of fabric 106 defined by cells (such as n-sided cells 110 of FIG. 1B) can be ‘masked’ using masks 190 to prevent painting the fabric 106 with binder material. Masks can be attached to each other to allow masking of a large portion (or all) of a major surface of the stent.

### General Discussion

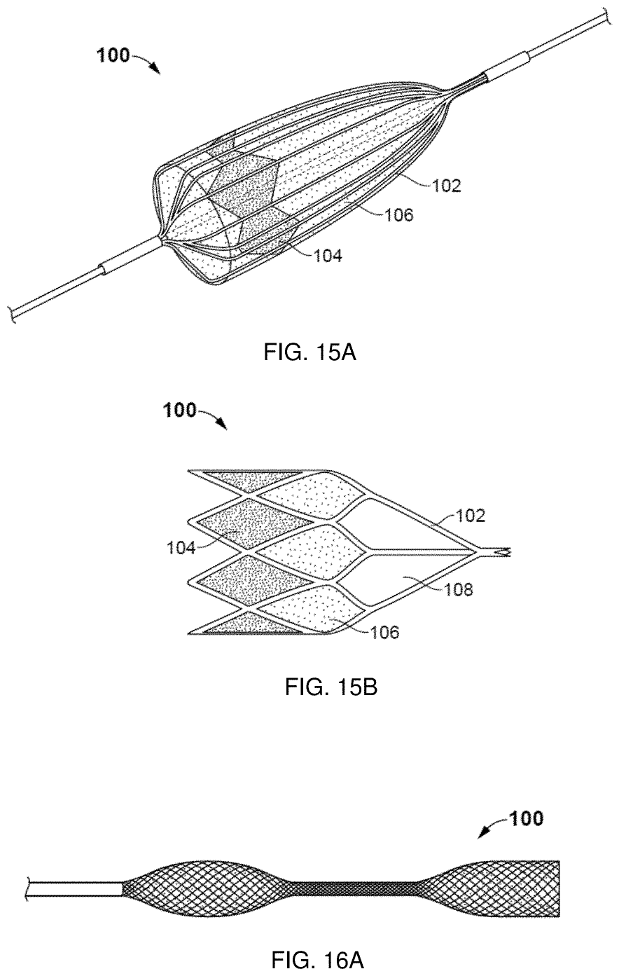

FIGS. 15A and 15B illustrate examples for attaching the fabric material 106 to the medical stent 101, according to embodiments. In an embodiment, some portion of the stent 101 could be covered by polymer film or layer 104, some portion of the stent 101 could be covered by fabric material 106, and further some portion of the stent 101 could be open 108, i.e., not covered with any material.

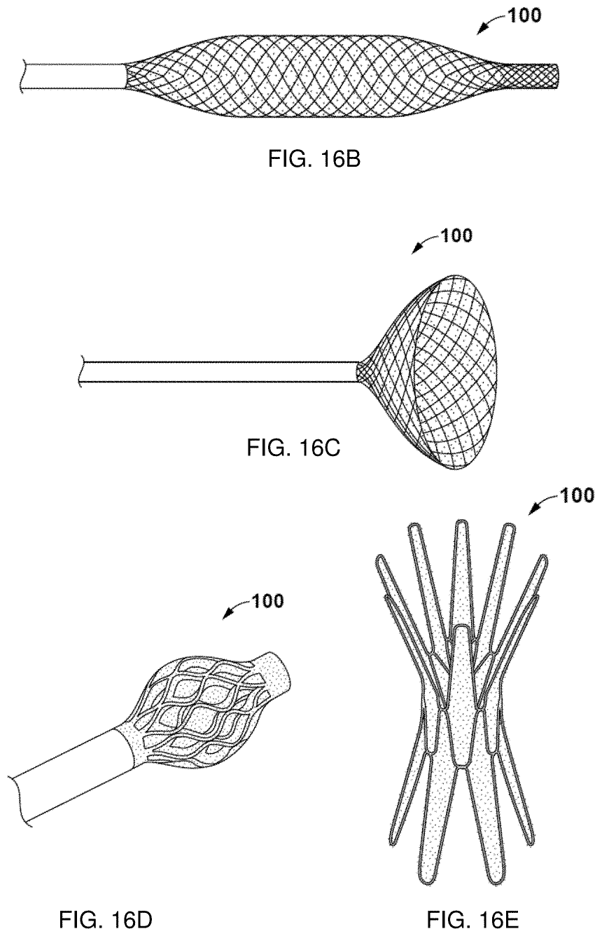

FIGS. 16A-16E illustrate examples of different shapes and designs of stent assemblies 100 incorporating embodiments of the present invention. As seen, the stent assembly 100 can be configured in any desirable shape, and is not limited to conical or cylindrical/tubular shapes.

According to the present invention, the disclosed methods for attaching the fabric material 106 to the medical stent, is a simple and inexpensive process. These methods obviate the requirement of complicated sewing or stitching process for the tiny struts 102 of the stent assembly 100. Further, customized fabric or fiber material 106 could be used along with desired polymers, for manufacturing a high-quality stent assembly 100.

All method steps described herein may be combined, in whole or in part, with any others, and may be performed in any order.

It will be obvious to the skilled artisan that the function of pores in a fabric (e.g., stent sleeve or graft) might be described differently in the case of a woven fabric, where the inter-fiber spaces of the woven fabric play the part of pores in a porous fabric.

The present invention has been described using detailed descriptions of embodiments thereof that are provided by way of example and are not intended to limit the scope of the invention. The described embodiments comprise different features, not all of which are required in all embodiments of the invention. Some embodiments of the present invention utilize only some of the features or possible combinations of the features. Variations of embodiments of the present invention that are described and embodiments of the present invention comprising different combinations of features noted in the described embodiments will occur to persons skilled in the art to which the invention pertains.

In the description and claims of the present disclosure, each of the verbs, “comprise”, “include” and “have”, and conjugates thereof, are used to indicate that the object or objects of the verb are not necessarily a complete listing of members, components, elements or parts of the subject or subjects of the verb. As used herein, the singular form “a”, “an” and “the” include plural references unless the context clearly dictates otherwise. For example, the term “a marking” or “at least one marking” may include a plurality of markings.

1. A stent assembly comprising:
a. a stent formed by a network of struts, the stent having an internal surface and an external surface; and
b. a porous fabric material covering at least a portion of at least one of the internal surface and the external surface, and bonded to the stent by a polymer binder that mediates between the struts and the fabric material to bind therebetween,
wherein:
i. at least 90%, by length, of the combined lengths of the struts of the network within a first surface region of the stent, is coated with the polymer binder,
ii. at least 70%, by area, of the fabric material disposed (A) within each one of one or more stent-area portions within a second surface region of the stent and (B) no more than 0.5 mm from a nearest respective strut, is rendered non-porous by a presence of the polymer binder within pores of the fabric material,
iii. at least 70%, by area, of portions of the fabric material (A) disposed within each one of said one or more stent-area portions and within said second surface region and (B) distanced at least 3 mm from a nearest respective strut, is characterized by pores that are free of the polymer binder, and
iv. the thickness of the polymer binder is not less than 1 micron and not greater than 70 microns.

2. The stent assembly of claim 1, wherein (i) the network of struts comprises a plurality of strut segments defined by intersection locations, (ii) an n-sided cell comprises n strut segments defined by n intersection locations, where n is an integer equal to at least 3 and at most 6, and (iii) the first surface region includes at least one n-sided cell.

3. The stent assembly of claim 2, wherein the first surface region includes at least 10 n-sided cells.

4. The stent assembly of claim 1, wherein (i) the network of struts includes a plurality of undulating rings of strut segments defined by bends and (ii) the first surface region includes at least one of the undulating rings.

5. The stent assembly of claim 4, wherein the first surface region includes at least 5 of the undulating rings.

6. The stent assembly of claim 1, wherein the first surface region includes at least 90% of the surface area of the stent.

7. The stent assembly of claim 1, wherein (i) the network of struts comprises a plurality of strut segments defined by intersection locations, (ii) the stent-area portions comprise n-sided cells each comprising n strut segments defined by n intersection locations, where n is an integer equal to at least 3 and at most 6, and (iii) the second surface region includes at least one stent-area portion.

8. The stent assembly of claim 7, wherein the second surface region includes at least 10 stent-area portions.

9. The stent assembly of claim 1, wherein (i) the network of struts includes is characterized by undulating rings of strut segments defined by bends, (ii) the stent-area portions comprise undulating rings, and (iii) the second surface region includes at least one stent-area portion.

10. (canceled)

11. The stent assembly of claim 1, wherein the second surface region includes at least 90% of the surface area of the stent.

12. The stent assembly of claim 1, wherein the first and second surface regions are substantially the same surface region.

13. (canceled)

14. (canceled)

15. (canceled)

16. The stent assembly of claim 1, wherein the thickness of the polymer binder is not less than 5 mm and not greater than 40 mm.

17. The stent assembly of claim 1, wherein at least 70%, by area, of the fabric material disposed (A) within each one of one or more stent-area portions within a second surface region of the stent and (B) no more than 1 mm from a nearest respective strut, are rendered non-porous by a presence of the polymer binder within pores of the fabric material

18. The stent assembly of claim 1, wherein at least 70%, by area, of the fabric material disposed (A) within each one of one or more stent-area portions within a second surface region of the stent and (B) no more than 2 mm from a nearest respective strut, are rendered non-porous by a presence of the polymer binder within pores of the fabric material

19. The stent assembly of claim 1, wherein at least 70%, by area, of portions of the fabric material (A) disposed within each one of said one or more stent-area portions and within said second surface region and (B) distanced at least 2 mm from a nearest respective strut, is characterized by pores that are free of the polymer binder.

20. The stent assembly of claim 1, wherein at least 70%, by area, of portions of the fabric material (A) disposed within each one of said one or more stent-area portions and within said second surface region and (B) distanced at least 3 mm from a nearest respective strut, is characterized by pores that are free of the polymer binder.

21. The stent assembly of claim 1, wherein (i) the binder coating forms a pair of binder-coating wings extending laterally in respective opposite directions from each of a plurality of the binder-coated struts, and (ii) the bonding of the fabric to the surface of the stent includes bonding the fabric to at least part of each binder wing.

22. A method for attaching a fabric material to a stent comprising a metal alloy, the stent formed by a network of struts and having an external surface and an internal surface, the method comprising:
a. engaging a porous fabric material with at least some of the surfaces of the struts; and
b. applying a polymer binder so as to bond the porous fabric material with said at least some of the surfaces of the struts, the applying being such that:
i. at least 90%, by length, of the combined lengths of the struts of the network within a first surface region of the stent, are bonded to the porous fabric material by polymer binder,
ii. at least 70%, by area, of the fabric material disposed (A) within each one of one or more stent-area portions within a second surface region of the stent and (B) no more than 0.5 mm from a nearest respective strut, is rendered non-porous by a presence of the polymer binder within pores of the fabric material,
iii. at least 70%, by area, of portions of the fabric material (A) disposed within each one of said one or more stent-area portions and within said second surface region and (B) distanced at least 3 mm from a nearest respective strut, is characterized by pores that are free of the polymer binder, and
iv. the thickness of the polymer binder is not less than 1 micron and not greater than 70 microns.

23. (canceled)

24. (canceled)

25. (canceled)

26. (canceled)

27. (canceled)

28. (canceled)

29. (canceled)

30. (canceled)

31. (canceled)

32. (canceled)

33. (canceled)

34. (canceled)

35. (canceled)

36. (canceled)

37. (canceled)

38. (canceled)

39. (canceled)

40. (canceled)

41. (canceled)

42. (canceled)

43. A radially compressible covered stent assembly comprising first and second stent-assembly segments displaced from one another longitudinally, the stent assembly having an external surface and an internal surface, the stent comprising:
a. a radially compressible stent formed by a network of struts, the network having a plurality of intersection locations at which intersections and/or bends define strut segments, each strut segment having respective outward-facing and inward facing-surfaces which correspond to the external and internal surfaces of the stent assembly, and two respective laterally-facing surfaces;
b. a polymer-binder coating applied to the struts so as to at least partially coat at least some of the strut segments and at least some of the intersection locations; and
c. a fabric covering at least a portion of at least one of the internal surface and the external surface so as to be in contact with the polymer-binder coating, at least 80% of said contact being characterized by the polymer-binder coating forming a bond between the fabric and a strut segment or intersection location at the respective point of contact,
wherein (i) first and second stent-assembly segments are at least partially coated with the polymer-binder coating with respective first and second coating thicknesses on one or both laterally-facing surfaces of at least some respective strut segments at or adjacent to respective intersection locations, said first and second coating thicknesses being different from each other, and (ii) the radial strength of the first stent-assembly segment is at least 20% greater than the radial strength of the second stent-assembly segment.

44. (canceled)

45. (canceled)

46. (canceled)

47. (canceled)

48. (canceled)

49. (canceled)

50. (canceled)

51. (canceled)

52. (canceled)

53. (canceled)

54. The stent assembly of claim 43, wherein the radial strength of the first stent-assembly segment is at least 50% greater than the radial strength of the second stent-assembly segment, wherein the radial strength of the first stent-assembly segment is at least 80% greater than the radial strength of the second stent-assembly segment.

55. (canceled)

56-87. (canceled)
